SEQUENCE LISTING (7 sequences)

SEQ ID NO: 1 - RNA, 21 nt, Artificial Sequence
  miRNA-23a
aucacauugccagggauuucc

SEQ ID NO: 2 - RNA, 22 nt, Artificial Sequence
  miRNA-24-1
ugccuacugagcugauaucagu

SEQ ID NO: 3 - RNA, 22 nt, Artificial Sequence
  miRNA-21
uagcuuaucagacugauguuga

SEQ ID NO: 4 - RNA, 21 nt, Artificial Sequence
  miRNA-499
uuaagacuugcagugauguuu

SEQ ID NO: 5 - RNA, 22 nt, Artificial Sequence
  miRNA-1
uggaauguaaagaaguguguau

SEQ ID NO: 6 - RNA, 22 nt, Artificial Sequence
  miRNA-133a
uuugguccccuucaaccagcug

SEQ ID NO: 7 - RNA, 18 nt, Artificial Sequence
  miRNA-208
auaagacgagcaaaaagc

(19) United States
(12) Patent Application Publication
(10) Pub. No.: US 2014/0242578 A1
(43) Pub. Date: Aug. 28, 2014

(54) SIMULTANEOUS DETECTION OF MULTIPLE MIRNAS USING CAPILLARY ELECTROPHORESIS SYSTEM EQUIPPED WITH PLURAL LASER-INDUCED FLUORESCENCE DETECTORS

(71) Applicant: KOREA INSTITUTE OF SCIENCE AND TECHNOLOGY, Seoul (KR)

(72) Inventors: Eun Joo SONG, Seoul (KR); Eunmi Ban, Seoul (KR)

(73) Assignee: KOREA INSTITUTE OF SCIENCE AND TECHNOLOGY, Seoul (KR)

(21) Appl. No.: 13/865,745

(22) Filed: Apr. 18, 2013

(30) Foreign Application Priority Data
Feb. 25, 2013 (KR) 10-2013-0020062

(51) Int. Cl. C12Q 1/68

(52) U.S. Cl. CPC C12Q 1/6883
USPC 435/6.11

(57) ABSTRACT

The present invention relates to a method for simultaneously detecting multiple miRNAs present in a sample and a kit for detecting same. According to the present invention, two or more miRNAs existing in trace amounts in a sample can be analyzed through only one measurement. The detection method of the present invention may be used for fast diagnosis of various diseases wherein miRNAs are involved, for example, cardiovascular diseases including myocardial infarction, with high accuracy.

### CROSS-REFERENCE TO RELATED APPLICATIONS

This application claims priority under 35 U.S.C. §119 to Korean Patent Application No. 10-2013-0020062, filed on Feb. 25, 2013, in the Korean Intellectual Property Office, the disclosure of which is incorporated herein by reference in its entirety.

### BACKGROUND

(a) Technical Field

The present invention relates to a method for simultaneously detecting multiple miRNAs present in a sample in trace amounts using a capillary electrophoresis system equipped with a plurality of detectors capable of detecting fluorescent materials at different wavelengths and a kit for detecting same.

(b) Background Art

A microRNA (miRNA) is a very short non-coding RNA consisting of 21-22 nucleotides on average. By regulating other genes through inhibition of translation of mRNA, it controls cellular differentiation, embryogenesis, metabolism and oncogenesis. It is thought that about 30% of the total genes of the human genome are regulated by miRNAs. The miRNAs are generated through transcription of individual genes in the non-coding regions. The miRNA is transcribed from a pri-miRNA which is a precursor transcribed in the nucleus by RNA polymerase II. The pri-miRNA is cleaved by the RNase III enzyme called Drosha (dsRNA-specific ribonuclease) to produce a pre-miRNA having a hairpin loop structure. The hairpin loop of the pre-miRNA is exported out of the nucleus by the proteins exportin-5 and Ran-GTP, which serve as cofactors, and processed into a miRNA duplex about 22 nucleotides in length by the action of the RNase III enzyme Dicer and TRBP (transactivation-responsive RNA binding protein). The miRNA duplex binds with RISC (RNA-induced silencing complex) and regulates genes by cleaving mRNAs or preventing translation.

Various kinds of miRNAs and target genes regulated thereby may be useful in predicting the mechanisms of various diseases. Since abnormally increased or decreased miRNA expression is observed in various diseases such as cancer, diabetes and cardiovascular diseases, the miRNA is recognized as a biomarker for diagnosing and predicting diseases.

In particular, research on miRNA as a biomarker for diagnosis and prediction of cardiovascular diseases has been very active. Since the cardiovascular diseases are a major cause of death worldwide and need quick treatment, useful biomarkers for fast and accurate diagnosis are required. Considering that miRNAs are found in various biological substances including cells, serum, blood plasma, saliva, tears and urine, and that the concentration of specific miRNAs differs between patients and healthy people, the miRNAs may be used as biomarkers for various diseases including the cardiovascular diseases.

However, since one miRNA is associated with various diseases, it is necessary to detect candidate diagnostic markers of several miRNAs simultaneously in order to minimize diagnostic error. Not only for diagnosis, but screening of several miRNAs is also necessary for discovery of new diagnostic markers. At present, microarray techniques and reverse transcription polymerase chain reaction techniques are mainly used for miRNA detection. Although the microarray technique allows detection of many kinds of miRNAs, quantitativity is not so good. Although the reverse transcription polymerase chain reaction technique is an excellent quantitative analysis method, it is costly, is problematic in reproducibility and, above all, requires a very long analysis time of 3-4 hours or longer. In this regard, the capillary electrophoresis technique is researched a lot because of the advantages of excellent quantitativity, reproducibility and resolution and, above all, short analysis time of less than 1 hour. Therefore, many researchers are striving to develop a method for detecting multiple miRNAs in short time with superior quantitativity, reproducibility and resolution, a satisfactory solution has not been achieved yet.

Throughout the specification, a number of publications and patent documents are referred to and cited. The disclosure of the cited publications and patent documents is incorporated herein by reference in its entirety to more clearly describe the state of the related art and the present invention.

### SUMMARY

The inventors of the present invention have made efforts to establish a method for simultaneously detecting multiple miRNAs associated with a disease. As a result, they have confirmed that multiple miRNAs existing in a cell can be detected with high sensitivity and speed by hybridizing the miRNAs using probes bearing different fluorescent materials and detecting them using a capillary electrophoresis system equipped with a plurality of detectors.

Accordingly, the present invention is directed to providing a method for simultaneously detecting two or more miRNAs present in a sample.

The present invention is also directed to providing a kit for simultaneously detecting two or more miRNAs for use in a capillary electrophoresis system equipped with a plurality of laser-induced fluorescence (LIF) detectors.

In an aspect, the present invention provides a method for simultaneously detecting two or more miRNAs present in a sample, including:

(a) extracting RNAs from a sample for analysis;

(b) hybridizing the extracted RNAs with single-stranded DNAs labeled with fluorescent materials as probes specific for two or more miRNAs expected to exist in the sample in a hybridization buffer;

(c) identifying DNA-miRNA complexes using a capillary electrophoresis system equipped with a plurality of laser-induced fluorescence (LIF) detectors; and

(d) identifying the presence of the two or more miRNAs in the sample by detecting peaks of the DNA-miRNA complexes at different wavelengths.

Other features and aspects of the present invention will be apparent from the following detailed description, drawings and claims.

### BRIEF DESCRIPTION OF THE DRAWINGS

The above and other objects, features and advantages of the present invention will now be described in detail with reference to certain exemplary embodiments thereof illustrated in the accompanying drawings which are given hereinbelow by way of illustration only, and thus are not limitative of the invention, and wherein:

FIG. 1 schematically describes generation of a miRNA in a cell and regulation of a gene mediated by the miRNA;

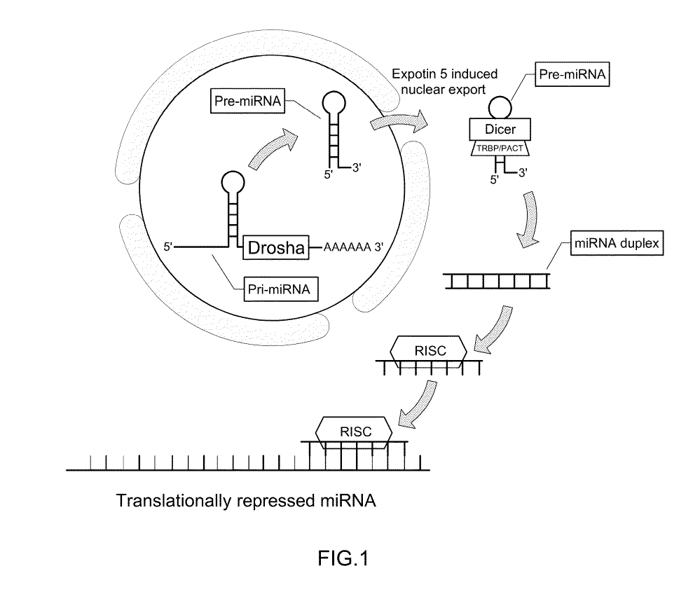

FIG. 2 describes a process of, for quantitative or qualitative analysis of multiple miRNAs, hybridizing target miRNAs with single-stranded DNAs specific for the target miRNAs and labeled with fluorescent residues of two different fluorescence wavelengths and identifying the presence of the target miRNAs simultaneously by detecting the peaks of unhybridized DNAs and DNA-target miRNA complexes by CE/dual LIF (CE/dLIF);

FIG. 3 shows a result of detecting single-stranded DNAs labeled with fluorescent residues of two different fluorescence wavelengths specific for miRNA-21, miRNA-23a and miRNA-24-1 by CE/dLIF and detecting DNA-miRNA complexes resulting from hybridization of the DNAs with miRNA-21, miRNA-23a and miRNA-24-1 by CE/dLIF;

FIG. 4 shows that, when miRNA-21, miRNA-23 and miRNA-24-1 are hybridized with single-stranded DNAs specific therefor and labeled with fluorescent residues, the intensity of the peaks of DNA-miRNA complexes increases linearly with the concentration of miRNA-21, miRNA-23 and miRNA-24-1; and

FIG. 5 shows a result of performing hybridization by adding single-stranded DNAs specific for miRNA-21, miRNA-23 and miRNA-24-1 and labeled with fluorescent residues to total RNAs extracted from H9c2 cardiomyocytes with or without miRNA-21, miRNA-23 and miRNA-24-1 added and identifying the peaks of unhybridized DNAs and DNA-miRNA-21 and DNA-miRNA-23 complexes by CE/dLIF, thereby detecting miRNA-21 and miRNA-23 in the H9c2 cardiomyocytes.

### DETAILED DESCRIPTION

Hereinafter, reference will now be made in detail to various embodiments of the present invention, examples of which are illustrated in the accompanying drawings and described below. While the invention will be described in conjunction with exemplary embodiments, it will be understood that the present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

The inventors of the present invention have made efforts to establish a method for simultaneously detecting multiple microRNAs (miRNAs) associated with a disease. As a result, they have confirmed that multiple miRNAs existing in a cell can be detected with high sensitivity and speed by hybridizing the miRNAs using probes bearing different fluorescent materials and detecting them using a capillary electrophoresis system equipped with a plurality of detectors.

The miRNA may have an important role in predicting various diseases. That is to say, since abnormally increased or decreased miRNA expression is observed in various diseases such as cancer, diabetes and cardiovascular diseases, the miRNA may be used as a biomarker for diagnosing and predicting of diseases. At present, microarray techniques and reverse transcription polymerase chain reaction techniques are frequently used for miRNA detection. Although the microarray technique allows detection of many kinds of miRNAs, quantitativity is very poor. The reverse transcription polymerase chain reaction technique is problematic in that it is costly, has reproducibility problem and, above all, requires a very long analysis time of 3-4 hours or longer. Although the capillary electrophoresis technique has the advantages of excellent quantitativity, reproducibility and resolution and, above all, short analysis time of less than 1 hour, it is difficult to detect various kinds of miRNAs simultaneously.

The detection method developed by the inventors of the present invention allows detection of various kinds of disease-related miRNAs present in a sample in trace amounts in short time, using a plurality of laser-induced fluorescence (LIF) detectors and is effectively applicable to diagnosis and prediction of a disease.

As used herein, the term “trace amount” refers to a small amount in the level of nanomolar, picomolar, femtomolar or less.

The extraction of total RNAs including miRNAs from a sample may be performed according to various methods known in the art. Specifically, TRIzol or Triton X-100 may be used for the extraction.

The detection method of the present invention may be used for diagnosis and prediction of various diseases, specifically for diagnosis of cardiovascular diseases including myocardial infarction.

Various miRNAs are known as biomarkers for cardiovascular diseases (Wilson et al., Dynamic microRNA expression programs during cardiac differentiation of human embryonic stem cells: role for miR-499, Circ Cardiovasc Genet, 3(5): 426-35 (2010)). Accordingly, when the detection method of the present invention is used for detection of the biomarkers for cardiovascular diseases, the miRNAs may be specifically two or more miRNAs selected from the group consisting of miRNA-23a, miRNA-24-1, miRNA-21, miRNA-499, miRNA-1, miRNA-133a and miRNA-208, more specifically two or more miRNAs selected from the group consisting of miRNA-23a of SEQ ID NO 1, miRNA-24-1 of SEQ ID NO 2, miRNA-21 of SEQ ID NO 3, miRNA-499 of SEQ ID NO 4, miRNA-1 of SEQ ID NO 5, miRNA-133a of SEQ ID NO 6 and miRNA-208 of SEQ ID NO 7, most specifically two or more miRNAs selected from the group consisting of miRNA-23a of SEQ ID NO 1, miRNA-24-1 of SEQ ID NO 2 and miRNA-21 of SEQ ID NO 3, as biomarkers for the cardiovascular diseases.

The miRNA may be present in trace amounts in various biological substances, including cells, serum, blood plasma, saliva, tears or urine. For example, when the miRNA is used as a biomarker for cardiovascular diseases, the cell may be a cardiomyocyte.

As used herein, the term “probe” refers to a naturally occurring or modified monomer or linear oligomer including a deoxyribonucleotide or a ribonucleotide that can be hybridized with a specific nucleotide sequence. Specifically, the probe may be single-stranded for maximizing hybridization efficiency. And, specifically, the probe may be a deoxyribonucleotide.

As the probe, not only a sequence perfectly complementary to the sequence including the miRNA but also one substantially complementary thereto may be used, as long as the specific hybridization is not interfered with.

The fluorescent material labeled at the probe may provide a signal allowing detection of the hybridization. The label may be attached to an oligonucleotide. The label that can be used in the present invention may be various fluorescent materials including fluorescein, phycoerythrin, rhodamine, lissamine, cyanine and the like. Specifically, 5′-carboxyfluorescein phosphoramidite (6-FAM), Cy3 or Cy5 may be used. The labeling may be performed according to the methods commonly employed in the art. For example, nick translation, random priming (Multiprime DNA labeling systems booklet, Amersham (1989)) or kination (Maxam & Gilbert, Methods in Enzymology, 65: 499 (1986)) method may be used.

An optimal hybridization condition may be determined by referring to Molecular Cloning, A Laboratory Manual (Joseph Sambrook, et al., Cold Spring Harbor Laboratory Press, Cold Spring Harbor, N.Y. (2001)) and Nucleic Acid Hybridization, A Practical Approach (Haymes, B. D., et al., IRL Press, Washington, D.C. (1985)). A stringent condition for hybridization may be determined by adjusting temperature, ionic strength (buffer concentration) and the presence of other compounds such as organic solvents. The stringent condition may be determined differently for different hybridized sequences. Various kinds of hybridization buffers commonly used in the related art may be used in the present invention. A buffer exhibiting the highest hybridization efficiency may be selected. For example, Tris-acetate buffer may be used as the hybridization buffer of the present invention.

In an exemplary embodiment of the present invention, the capillary electrophoresis system of the present invention may be equipped with a plurality of LIF detectors (i.e., a CE/LIF system).

The plurality of LIF detectors may be dual LIF (dLIF) or triple LIF (tLIF) detectors.

The wavelength of the LIF detector may be different depending on the fluorescent material to be detected. In case of dual LIF detectors, two fluorescent materials may be used to exhibit two excitation wavelengths or emission wavelengths. And, in case of triple LIF detectors, three fluorescent materials may be used to exhibit three excitation wavelengths or emission wavelengths.

The wavelengths may be different depending on the fluorescent material used and may be determined considering the detection target, purpose of detection and the fluorescent material used. The excitation wavelength may be specifically 400-700 nm, more specifically two or more selected from the group consisting of 488 nm, 514 nm, 530 nm, 560 nm, 594 nm, 635 nm, 640 nm and 685 nm. The emission wavelength may be specifically 500-800 nm.

For example, when 6-FAM is used as the fluorescent material, an LIF detector having an excitation wavelength of 488 nm may be used. And, when Cy-5 is used, an LIF detector having an excitation wavelength of 635 nm may be used.

If a capillary electrophoresis system equipped with dual or triple LIF detectors (CE/dLIF or CE/tLIF system) is used, two or more different miRNAs may be detected simultaneously at two or three different wavelengths.

In another aspect, the present invention provides a kit for simultaneously detecting two or more miRNAs for use in a capillary electrophoresis system equipped with a plurality of LIF detectors.

In an exemplary embodiment of the present invention, the kit of the present invention includes: (a) two or more probes that can be specifically hybridized with two or more miRNAs and two or more fluorescent materials that can be labeled at the probes, or two or more probes labeled with two or more fluorescent materials that can be specifically hybridized with the miRNAs; (b) a hybridization buffer; and (c) a buffer for separation of DNA-miRNA complexes.

The fluorescent material that can be used in the present invention may be various fluorescent materials known in the art. Specifically, two or more fluorescent materials selected from the group consisting of including fluorescein, phycoerythrin, rhodamine, lissamine and cyanine may be used. In an exemplary embodiment of the present invention, the hybridization buffer may be Tris-acetate buffer.

In an exemplary embodiment of the present invention, the separation buffer may be Tris-borate buffer.

The kit of the present invention may be used to simultaneously detect multiple miRNAs present in a sample. Specifically, it may be used to detect two or more miRNAs selected from the group consisting of miRNA-23a, miRNA-24-1, miRNA-21, miRNA-499, miRNA-1, miRNA-133a and miRNA-208.

### EXAMPLES

The present invention will be described in more detail through examples. The following examples are for illustrative purposes only and it will be apparent to those skilled in the art not that the scope of this invention is not limited by the examples.

### Example 1

### Identification of Peaks of Unhybridized DNAs and miRNA-DNA Complexes by Capillary Electrophoresis

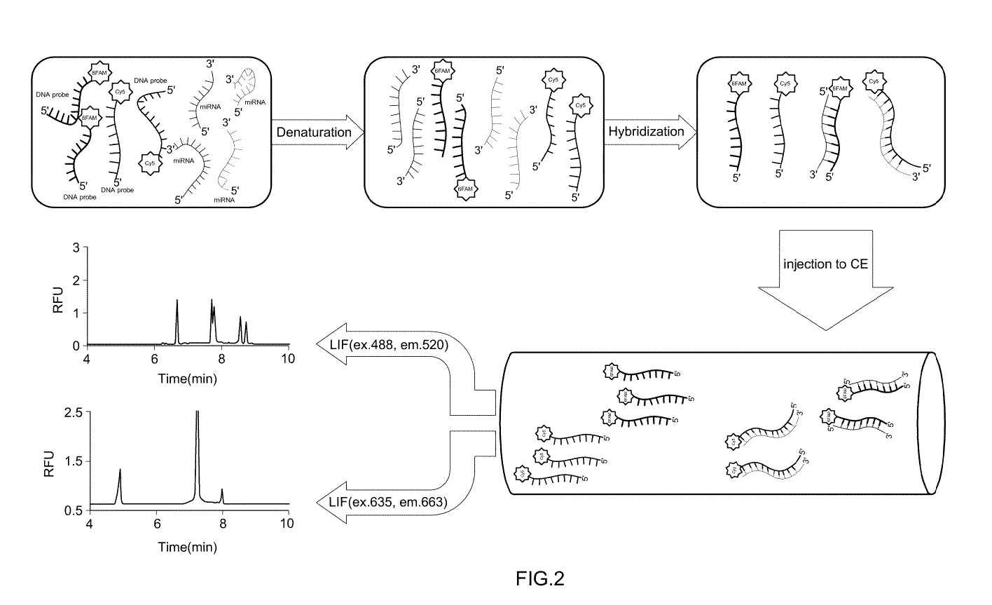

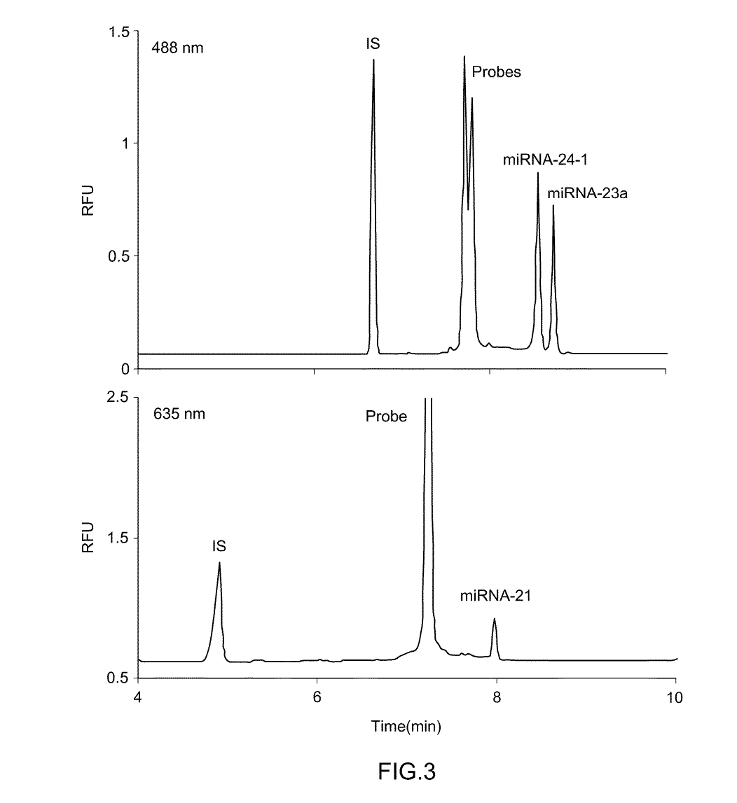

miRNA-23a, miRNA-24-1 and miRNA-21 were selected from the biomarker miRNAs for cardiovascular diseases. As described in FIG. 2, DNA probes labeled with the fluorescent material 5′-carboxyfluorescein phosphoramidite (6-FAM) specific for miRNA-23a (5′-AUCACAUUGCCAGGGAUUUCC-3′, SEQ ID NO 1) and miRNA-24-1 (5′-UGCCUACUGAGCUGAUAUCAGU-3′, SEQ ID NO 2) and a DNA probe labeled with the fluorescent material Cy-5 specific for miRNA-21 (5′-UAGCUUAUCAGACUGAUGUUGA-3′, SEQ ID NO 3) were denatured at 95° C. for 5 minutes along with miRNA-21, miRNA-23a and miRNA-24-1, respectively, hybridized at 40° C. for 15 minutes in 50 mM Tris-acetate buffer (pH 8.0) containing 0.1 mM EDTA, 50 mM NaCl and 1% Triton X-100, and then analyzed using a capillary electrophoresis (CE) system equipped with laser-induced fluorescence (LIF) detectors. FIG. 3 shows the peaks of the two unhybridized DNA probes and DNA-miRNA-23 and DNA-miRNA-24-1 complexes at 488 nm and those of the unhybridized DNA probe and a DNA-miRNA-21 complex at 635 nm. The CE system was PA 800 plus CE system (Beckman Coulter, Fullerton, Calif., USA) and the LIF detectors were Beckman P/ACE System Laser Module 488 and Laser Module 635 with excitation wavelengths of 488 nm and 635 nm and emission wavelengths of 520 nm and 663 nm, respectively. The DNA-miRNA complexes were separated in 100 mM Tris-borate buffer (pH 10.0) by applying a voltage of 14 kV into an uncoated capillary (Beckman Coulter) having an inner diameter of 75 μm and a length of 30 cm. Sample injection was carried out at 0.5 psi for 5 seconds.

### Example 2

### Confirmation of Quantitativity of miRNA Detection

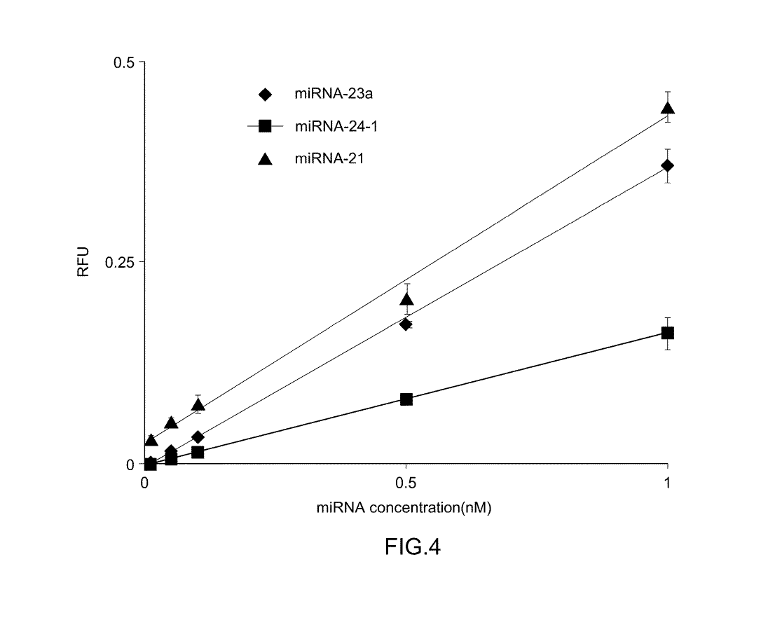

DNA probes labeled with the fluorescent materials 6-FAM or Cy-5, which are specific for miRNA-21, miRNA-23a and miRNA-24-1, were denatured at 95° C. for 5 minutes along with miRNA-21, miRNA-23a and miRNA-24-1 of 10 pM to 1 nM concentration, hybridized at 40° C. for 15 minutes in TEN hybridization buffer, and then analyzed using a CE system equipped with LIF detectors (FIG. 4). The analysis condition was the same as in Example 1. It was found out that the peak intensity of the DNA-miRNA complexes increases linearly with the concentration of the miRNAs and thus quantitative analysis is possible.

### Example 3

### Detection of miRNAs in Cardiomyocytes

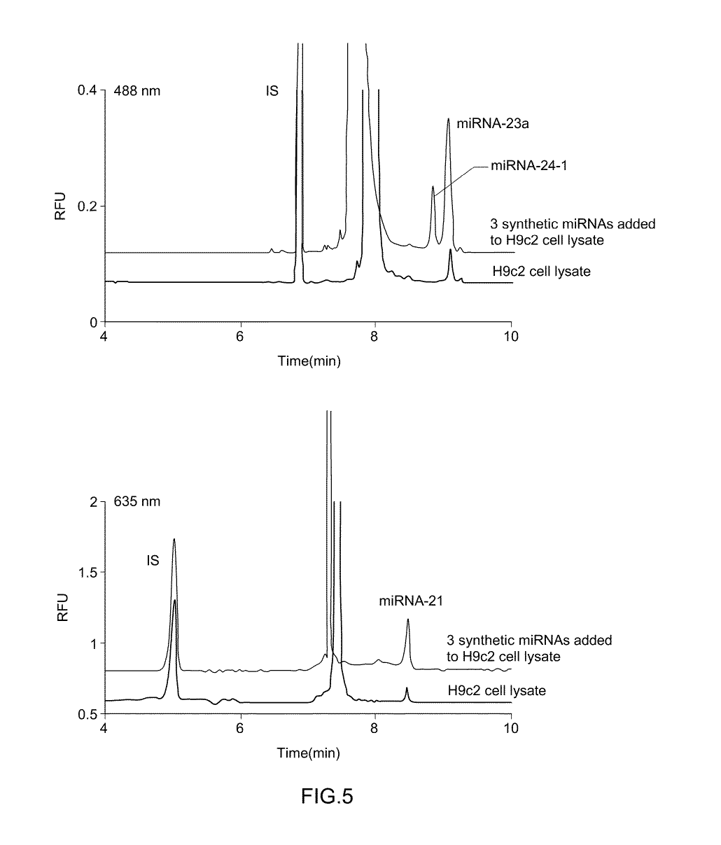

H9c2 cardiomyocytes (Korean Cell Line Bank) were cultured in DMEM containing 10% FBS and 1 vol % penicillin-streptomycin. The medium was replaced every other day and an incubator used to culture the cells was maintained at 37° C. and 5% CO₂. Total RNAs were extracted from 1×10⁶ cells on a 100-mm dish using TRIzol and Triton X-100 with or without adding 1 nM miRNA-21, miRNA-23a and miRNA-24-1. The extracted total RNAs and DNA probes specific for miRNA-21, miRNA-23a and miRNA-24-1 were denatured at 95° C. for 5 minutes, hybridized at 40° C. for 15 minutes in 50 mM Tris-acetate buffer (pH 8.0) containing 0.1 mM EDTA, 50 mM NaCl and 1% Triton X-100, and then analyzed using a CE system equipped with dual LIF detectors (CE/dLIF). The CE system was PA 800 plus CE system (Beckman Coulter, Fullerton, Calif., USA) and the LIF detectors were Beckman P/ACE System Laser Module 488 and Laser Module 635 with excitation wavelengths of 488 nm and 635 nm and emission wavelengths of 520 nm and 663 nm, respectively. The DNA-miRNA complexes were separated in 100 mM Tris-borate buffer (pH 10.0) by applying a voltage of 14 kV into an uncoated capillary (Beckman Coulter) having an inner diameter of 75 μm and a length of 30 cm. Sample injection was carried out at 0.5 psi for 5 seconds. For the cell extract to which 1 nM miRNA-21, miRNA-23a and miRNA-24-1 were added, the peaks of DNA-miRNA-23a and DNA-miRNA-24-1 complexes could be identified at 488 nm could be identified at 488 nm and that of a DNA-miRNA-21 complex could be identified at 635 nm (FIG. 5). And, from the cell extract to which no miRNA was added, the peak of the DNA-miRNA-23a complex was detected at 488 nm and that of the DNA-miRNA-21 complex was detected at 635 nm.

The features and advantages of the present disclosure may be summarized as follows:

(i) The present invention provides a method for detecting two or more miRNAs simultaneously and a kit for detecting same.

(ii) In accordance with the present invention, two or more miRNAs existing in trace amounts in a sample can be analyzed through only one measurement.

(iii) The detection method of the present invention may be used for fast diagnosis of various diseases wherein miRNAs are involved, for example, cardiovascular diseases including myocardial infarction, with high accuracy.

The present invention has been described in detail with reference to specific embodiments thereof. However, it will be appreciated by those skilled in the art that various changes and modifications may be made in these embodiments without departing from the principles and spirit of the invention, the scope of which is defined in the appended claims and their equivalents.

What is claimed is:

1. A method for simultaneously detecting two or more miRNAs present in a sample, comprising:
extracting RNAs from a sample to be analyzed;
hybridizing the extracted RNAs with single-stranded DNAs labeled with fluorescent materials as probes specific for two or more miRNAs expected to exist in the sample in a hybridization buffer;
identifying DNA-miRNA complexes using a capillary electrophoresis system equipped with a plurality of laser-induced fluorescence (LIF) detectors; and
detecting the two or more miRNAs existing in the sample by detecting peaks of the DNA-miRNA complexes at different wavelengths.

2. The detection method according to claim 1, wherein the sample is a cell, serum, blood plasma, saliva, tear or urine.

3. The detection method according to claim 2, wherein the cell is a cardiomyocytes.

4. The detection method according to claim 1, wherein the two or more miRNAs are two or more miRNAs selected from the group consisting of miRNA-23a, miRNA-24-1, miRNA-21, miRNA-499, miRNA-1, miRNA-133a and miRNA-208.

5. The detection method according to claim 4, wherein the two or more miRNAs are two or more miRNAs selected from the group consisting of miRNA-23a of SEQ ID NO 1, miRNA-24-1 of SEQ ID NO 2 and miRNA-21 of SEQ ID NO 3.

6. The detection method according to claim 1, wherein the fluorescent materials are two or more fluorescent materials selected from the group consisting of fluorescein, phycoerythrin, rhodamine, lissamine and cyanine.

7. The detection method according to claim 6, wherein the fluorescein is 5′-carboxyfluorescein phosphoramidite (6-FAM).

8. The detection method according to claim 6, wherein the cyanine is Cy3 or Cy5.

9. The detection method according to claim 1, wherein the plurality of LIF detectors have different excitation wavelengths and emission wavelengths.

10. The detection method according to claim 9, wherein the excitation wavelengths range from 400 to 700 nm.

11. The detection method according to claim 10, wherein the excitation wavelengths are two or more selected from the group consisting of 488 nm, 514 nm, 530 nm, 560 nm, 594 nm, 635 nm, 640 nm and 685 nm.

12. The detection method according to claim 1, wherein the plurality of LIF detectors are dual LIF detectors (dual LIF) in which two LIF detectors are combined.

13. The detection method according to claim 1, wherein said detecting the two or more miRNAs comprises identifying the peaks of the two or more DNA-miRNA complexes resulting from hybridization with DNAs labeled with different fluorescent materials at different wavelengths.

14. A kit for simultaneously detecting two or more miRNAs for use in a capillary electrophoresis system equipped with a plurality of laser-induced fluorescence (LIF) detectors, comprising: two or more probes that can be specifically hybridized with two or more miRNAs and two or more fluorescent materials that can be labeled at the probes, or two or more probes labeled with two or more fluorescent materials that can be specifically hybridized with the miRNAs; a hybridization buffer; and a buffer for separation of DNA-miRNA complexes.

15. The kit for simultaneously detecting two or more miRNAs according to claim 14, wherein the fluorescent materials are two or more fluorescent materials selected from the group consisting of fluorescein, phycoerythrin, rhodamine, lissamine and cyanine.

16. The kit for simultaneously detecting two or more miRNAs according to claim 14, wherein the hybridization buffer is Tris-acetate buffer.

17. The kit for simultaneously detecting two or more miRNAs according to claim 14, wherein the buffer for separation of DNA-miRNA complexes is Tris-borate buffer.

18. The kit for simultaneously detecting two or more miRNAs according to claim 14, wherein the two or more miRNAs are two or more miRNAs selected from the group consisting of miRNA-23a, miRNA-24-1, miRNA-21, miRNA-499, miRNA-1, miRNA-133a and miRNA-208.
